(19) United States
(12) Patent Application Publication
(10) Pub. No.: US 2003/0153594 A1
(43) Pub. Date: Aug. 14, 2003

(54) Use of particular compounds for prophylaxis and treatment of hepatitis c

(76) Inventors: Lutz Webber, Grenzach-Wyhlen (DE); Sven Nerdinger, Unterhaching (DE); Michael W Cappi, Munchen (DE); Dirk Behnke, Planegg (DE)

Correspondence Address: EDWARDS & ANGELL, LLP, P.O. BOX 9169, BOSTON, MA, 02209, US

(21) Appl. No.: 10/181,515

(22) PCT Filed: Jan. 15, 2001
(86) PCT No.: PCT/EP01/00390

(51) Int. Cl. A61K 31/4745; A61K 31/47; A61K 31/12

(52) U.S. Cl. 514/300; 514/312; 514/682

(57) ABSTRACT

The present invention relates to the use of a compound of formula (I):

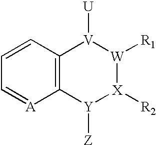

wherein:

U—V—W(R₁)—X(R₂)—Y—Z has the formula RHN—C═CR₁—CR₂═C—OH, HO—C═CR₁—CR₂═C—OH or O═C—CR₁═CR₂—C═O,

A is an N atom or the group CH,

R is an H atom, a C₁-C₆alkyl group or a benzyl group,

R₁ is an H atom or a methyl group, and

R₂ is an H atom or a group of formula

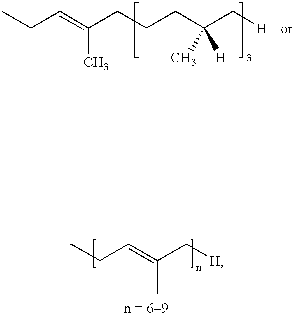

for therapy or prophylaxis of hepatitis C.

The present invention relates to the use of specific compounds for prophylaxis and therapy of hepatitis C.

Hepatitis C is a chronic viral infection of epidemic proportions. 400 million people, or every fifteenth person world-wide, and about 800,000 Germans suffer from this disease; in Germany, there are approximately 40,000 new cases per year.

60-80% of infections with the hepatitis C virus (HCV) become chronic. In the course of 10-20 years, 20-30% of patients develop cirrhosis of the liver; 20-25 years after infection, hepatocellular carcinomas are a frequent occurrence. It is currently estimated that about 20% of all affected persons will develop life-threatening liver conditions. The number of deaths caused by hepatitis C has increased tenfold in the last ten years; hepatitis C is now one of the ten most common causes of death in Germany. Treating hepatitis C with medicaments has hitherto been successful to only a limited extent.

The problem of the present invention was accordingly to make available active ingredients for use in the prophylaxis and/or therapy of hepatitis C.

The problem is solved by the use of a compound of formula (I):

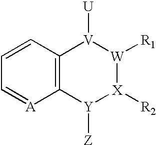

wherein:

U—V—W(R₁)—X(R₂)—Y—Z has the formula RHN—C═CR₁—CR₂═C—OH, HO—C═CR₁—CR₂═C—OH or O═C—CR₁═CR₂—C═O,

A is an N atom or the group CH,

R is an H atom, a C₁-C₆alkyl group or a benzyl group,

R₁ is an H atom or a methyl group, and

R₂ is an H atom or a group of formula

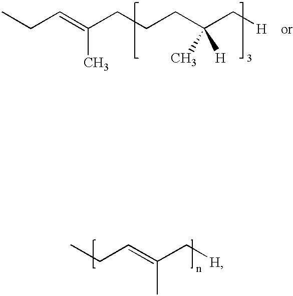

n being 6, 7, 8 or 9,

for therapy or prophylaxis of hepatitis C.

n is preferably 7.

The alkyl radicals are preferably a methyl, ethyl, propyl, isopropyl or butyl group.

In accordance with one embodiment, compounds are used wherein group U—V—W(R₁)—X(R₂)—Y—Z has the formula RHN—C═CR₁—CR₂═C—OH

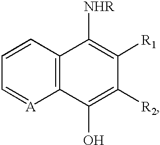

the formula HO—C═CR₁—CR₂═C—OH:

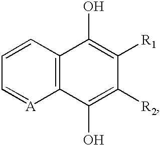

or the formula O═C—CR₁═CR₂—C═O:

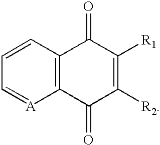

A is preferably a CH group:

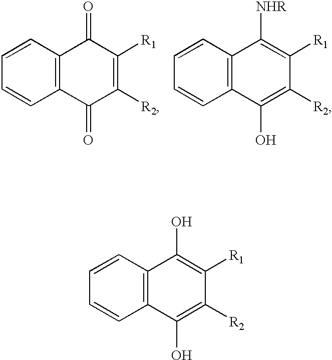

or an N atom:

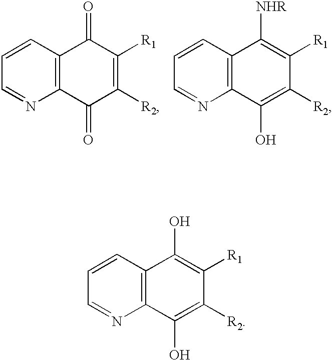

According to an especially preferred embodiment, R is an H atom or a benzyl group.

Special preference is given to the use of compounds of formula:

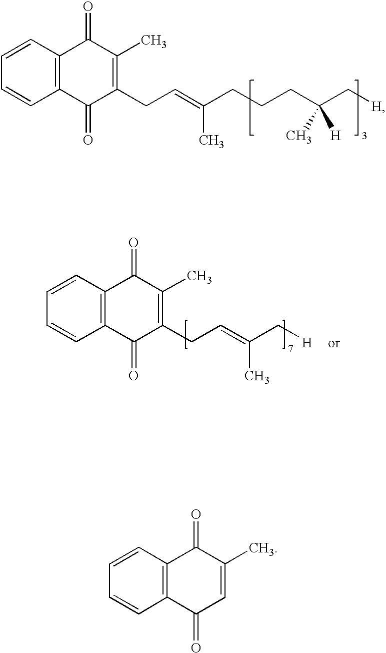

Such compounds possess especially good activity in the prophylaxis or combating (therapy) of hepatitis C (viruses).

For example, the compounds can be administered orally, parenterally, transdermally, intranasally, transmucosally, systemically, subcutaneously, intravascularly, intramuscularly, topically, rectally, intravaginally and, for example, can be formulated as a capsule, tablet, pastille, lozenge, spray, nasal spray, pessary, suppository, injection preparation, enema, ointment, cream or patch.

The compounds can also be formulated as delayed-release preparations. The active ingredients according to the invention can be used as prophylactic agents or therapeutic agents in human and veterinary medicine.

They can be used locally or systemically. Systemic administration is understood to mean, for example, intravenous, intrapleural, intraperitoneal, rectal or oral administration or irrigation of body cavities and the urinary bladder. Local administration is understood to mean, for example, subcutaneous, intracutaneous, intratumoral or peritumoral administration, for example in the form of injection solutions, injection suspensions, creams, lotions, gels and ointments.

On systemic and local administration, the active ingredients used according to the invention have a dose-dependent HCV-combating action. When used therapeutically, the daily dose of active ingredients according to the invention is of the order of from 0.01 to 100 mg/kg of bodyweight, preferably from 2 to 40 mg/kg of bodyweight. In individual cases, the dosage may be higher or lower than that mentioned above.

The active ingredients used according to the invention can be used in known manner—depending upon the individual clinical entity—in a formulation, for example patches, ointments, pastes, creams, soluble powders, emulsions, powders, suspensions and injection solutions.

The active ingredients used according to the invention, for example in an injection solution, can be dissolved, where appropriate with the aid of solubilisers, in dilute physiologically acceptable bases and brought into an injectable form having a pH of from 6 to 8, especially from 6.9 to 7.5, by the addition of physiologically acceptable acids.

Examples of physiologically acceptable bases are hydroxides, hydrogen carbonates, carbonates of alkali and alkaline-earth metals, especially of potassium, sodium and calcium.

Examples of physiologically acceptable acids are lactic acid, citric acid, tartaric acid, oxalic acid, malic acid, acetic acid, formic acid, benzoic acid, salicylic acid, hydrochloric acid, sulphuric acid or phosphoric acid.

Excipients may be mixed in with the formulation of active ingredients used according to the invention (one or more of which may be used). Such non-toxic and pharmaceutically suitable excipients may be, for example, solid, semi-solid or liquid carriers, emulsifiers or dispersants. The concentration of active ingredients according to the invention is from 1 to 90% by weight, preferably from 5 to 50% by weight.

The dosage units of the active ingredients used according to the invention may consist of, for example, 1, 2, 3 or 4 individual doses or ½, ⅓ or ¼ of an individual dose. An individual dose preferably contains the amount of active ingredient given on one administration, which usually corresponds to all, a half or a third or even a quarter of a daily dose.

Creams, pastes, ointments and gels may comprise, beside the active ingredient(s), customary carriers known to the person skilled in the art, for example waxes, paraffins, starches, vegetable and animal fats, cellulose derivatives, tragacanth, silicic acid, talcum, zinc oxide, bentonites, silicones, polyethylene glycols.

Sprays and powders may comprise, besides the active ingredient(s), customary carriers known to the person skilled in the art, for example lactose, talcum, silicic acid, aluminium hydroxide, calcium silicate or polyamide powder or mixtures thereof. Sprays may comprise, in addition, propellants, for example chlorofluorocarbons.

Suppositories may comprise, besides the active ingredient(s), customary carriers known to the person skilled in the art, for example polyethylene glycols, fats or mixtures thereof.

Local administration of the active ingredients used according to the invention may be carried out by means of micro-machines.

In order to obtain better, locally relevant active ingredient concentrations and for greater tolerability, the active ingredients may be packed in liposomes.

If advantageous for treatment of the disease or for the general condition of the patient or of the patient's family, combinations with other active ingredients of use to the patient may be administered simultaneously or at different times.

The compounds used in accordance with the invention can be prepared in a manner customary per se.

### EXAMPLES

The IC₅₀ values were determined analogously to the NS3 proteinase assay described in: M. Taliani et al., Analytical Biochemistry, 240 (1996) 60-67. Evaluation was carried out using the “GraFit 4” program from Erithacus Software Ltd.

### Example 1

### Menadione

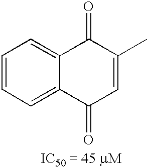

### Example 2

### 1,4-naphthoquinone

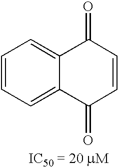

### Example 3

### 1-amino-4-naphthol

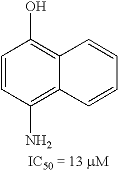

### Example 4

### 1-benzylamino-4-naphthol

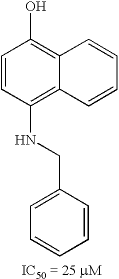

### Example 5

### 5-amino-8-hydroxyquinoline

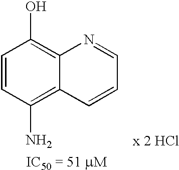

1. Use of a compound of formula (I): 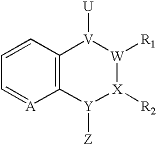
wherein:
U—V—W(R₁)—X(R₂)—Y—Z has the formula RHN—C═CR₁—CR₂═C—OH, HO-C═CR₁—CR₂═C—OH or O═C—CR₁═CR₂—C═O,
A is an N atom or the group CH,
R is an H atom, a C₁-C₆alkyl group or a benzyl group,
R₁ is an H atom or a methyl group, and
R₂ is an H atom or a group of formula 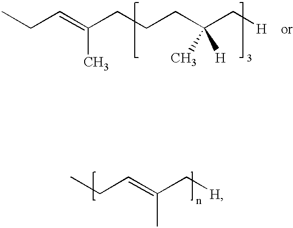
n being 6,7, 8 or 9,
for therapy or prophylaxis of hepatitis C.

2. Use according to claim 1, characterised in that U—V—W(R₁)—X(R₂)—Y—Z has the formula RHN—C═CR₁—CR₂═C—OH:

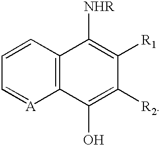

3. Use according to claim 1, characterised in that U—V—W(R₁)—X(R₂)—Y—Z has the formula HO—C═CR₁—CR₂═C—OH:

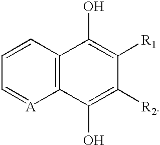

4. Use according to claim 1, characterised in that U—V—W(R₁)—X(R₂)—Y—Z has the formula O═C—CR₁═CR₂—C═O:

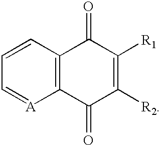

5. Use according to one of the preceding claims, characterised in that A is a CH group:

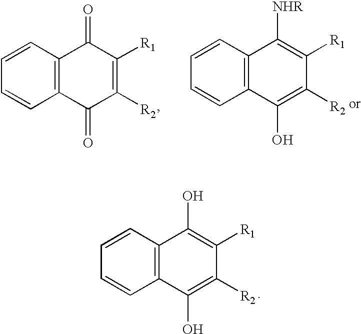

6. Use according to one of claims 1 to 4, characterised in that A is an N atom:

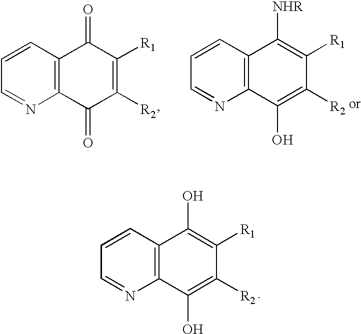

7. Use according to one of the preceding claims, characterised in that R is an H atom.

8. Use according to one of claims 1 to 6, characterised in that R is a benzyl group.

9. Use according to one of the preceding claims, characterised in that the compound has the formula:

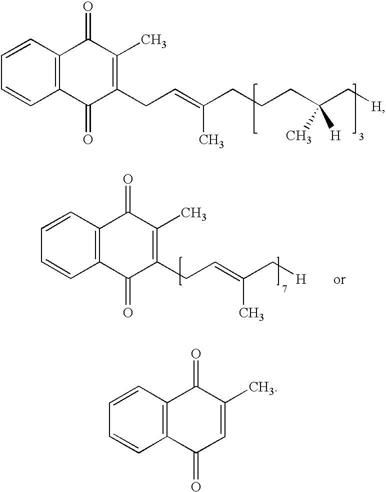

10. Use according to one of the preceding claims, characterised in that the compound is administered orally, parenterally, transdermally, intranasally, transmucosally, systemically, subcutaneously, intravascularly, intramuscularly, topically, rectally or intravaginally.

11. Use according to one of the preceding claims, characterised in that the compound is formulated as a capsule, tablet, pastille, lozenge, spray, nasal spray, pessary, suppository, injection preparation, enema, ointment, cream or patch.

12. Use according to one of the preceding claims, characterised in that the compound is formulated as a delayed-release preparation.

13. Use according to one of the preceding claims, characterised in that, in addition, a further active ingredient is used.
